(19) United States
(12) Patent Application Publication
(10) Pub. No.: US 2022/0410310 A1
(43) Pub. Date: Dec. 29, 2022

(54) ULTRASHORT PULSE LASER MARKING APPARATUS AND METHOD

(71) Applicant: Tri-Star Technologies, El Segundo, CA (US)

(72) Inventor: Igor Murokh, Santa Monica, CA (US)

(21) Appl. No.: 17/849,636

(22) Filed: Jun. 25, 2022

Related U.S. Application Data
(60) Provisional application No. 63/215,385, filed on Jun. 25, 2021.

(51) Int. Cl. B23K 26/0622; B23K 26/06; B23K 26/18

(52) U.S. Cl. CPC B23K 26/0624; B23K 26/0643; B23K 26/18

(57) ABSTRACT

Embodiments of an ultrashort pulse laser marking apparatus for forming indelible identifiers on discreet consumable articles, and corresponding methods, are disclosed. An ultrashort pulse laser transmission element of the apparatus is configured to transmit a beam of laser energy toward a marking zone to form an optically-readable indelible identifier on discrete consumable articles. The beam may have a pulse duration less than 10 picosecond, and a wavelength of less than 1.5 microns. The consumable articles may comprise a photoreactive pigment configured to undergo a color change upon exposure to the beam of laser energy, and the indelible identifier may be defined by the color change. Alternatively or in addition, the optical readability may be at least in part by way of a primary pattern reflected light intensity being distinguishable from a baseline reflected light intensity or from a secondary reflected light intensity from a viewpoint outward of the article.

### RELATED APPLICATIONS

This application claims the benefit of U.S. Provisional Application No. 63/215,385 filed Jun. 25, 2021, the contents which are incorporated by this reference in their entireties for all purposes as if fully set forth herein.

### TECHNICAL FIELD

The present disclosure relates to using laser energy to apply markings to discrete consumable articles such as pharmaceutical capsules.

### SUMMARY

Certain deficiencies of the prior art are overcome by the provision of ultrashort pulse laser marking apparatuses and methods as disclosed herein.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a diagrammatic view of one non-limiting example of an ultrashort pulse laser marking apparatus in accordance with the present disclosure, wherein the laser beam is steered by a beam steering element;

FIG. 2 is a diagrammatic view of one alternative non-limiting example of an ultrashort pulse laser marking apparatus in accordance with the present disclosure, wherein the laser beam is partially masked by way of a beam masking element;

FIG. 3 is a diagrammatic perspective view of a plurality of discreet consumable articles in the form of pharmaceutical capsules;

FIG. 4 is a diagrammatic top view of one non-limiting example of a marking zone, wherein a multiplicity of discreet consumable articles are retained within the marking zone for marking;

FIG. 5 is a diagrammatic top view of one non-limiting example of a discrete consumable article which has been marked with an indelible identifier, wherein indelible identifier includes multiple characters;

FIG. 6A is a diagrammatic magnified view of detail 6 in FIG. 5, representing a case in which the consumable article of FIG. 5 comprises a photoreactive pigment configured to undergo a color change upon exposure to a beam of laser energy, and the indelible identifier is defined by the color change;

FIG. 6B is a diagrammatic magnified view of detail 6 in FIG. 5, representing an alternate case in which the indelible identifier of FIG. 5 is defined at least by a primary pattern field having a primary array of primary light interface elements distributed therein;

FIG. 6C is a diagrammatic magnified view of detail 6 in FIG. 5, representing a further alternate case in which the indelible identifier of FIG. 5 is defined at least by a primary pattern field having a primary array of primary light interface elements distributed therein, and one or more secondary pattern fields having a secondary array of secondary light interface elements distributed therein;

FIG. 7A is a diagrammatic partial cross-sectional view taken along lines 7A-7A in FIG. 6A;

FIG. 7B is a diagrammatic partial cross-sectional view taken along lines 7B-7B in FIG. 6B;

FIG. 7C is a diagrammatic partial cross-sectional view taken along lines 7C-7C in FIG. 6C;

FIG. 8 is a diagrammatic partial cross-sectional view similar to that of FIG. 7B, but wherein the primary light interface elements have an alternate cross-sectional shape;

FIG. 9 is a diagrammatic side view of the example article illustrated in FIG. 5, illustrating a scenario in which the light source axis and the viewing axis are off-angle with respect to one another during the optical reading of the indelible identifier;

FIG. 10 is a chart and associated legend illustrating how varying degrees of light intensity contrasts between base reflection, low reflection and high reflection areas may be used to optically perceive character boundaries in an indelible identifier;

FIG. 11 is a diagrammatic cross-sectional view of an outward letter of one non-limiting example of a discreet consumable article, showing how individual light interface elements may act to block, refract and reflect light in the respective pattern field;

FIG. 12 is a diagrammatic top view illustrating how a optically-perceptible border-defining light intensity contrast may be produced based in part on the differential orientation of the light interface elements of adjacent pattern fields, and the respective orientation of the incoming light source and the viewpoint;

FIG. 13 is a diagrammatic graph illustrating an example pulse duration of a femtosecond laser;

FIG. 14 is a diagrammatic graph illustrating an example wavelength of a femtosecond laser; and

FIG. 15 is a diagrammatic block diagram representing one or more example implementations of a method of using an ultrashort pulse laser marking apparatus for forming indelible identifiers on discreet consumable articles.

### DETAILED DESCRIPTION OF THE PREFERRED EMBODIMENTS

Referring now to the drawings, like reference numerals designate identical or corresponding features throughout the several views.

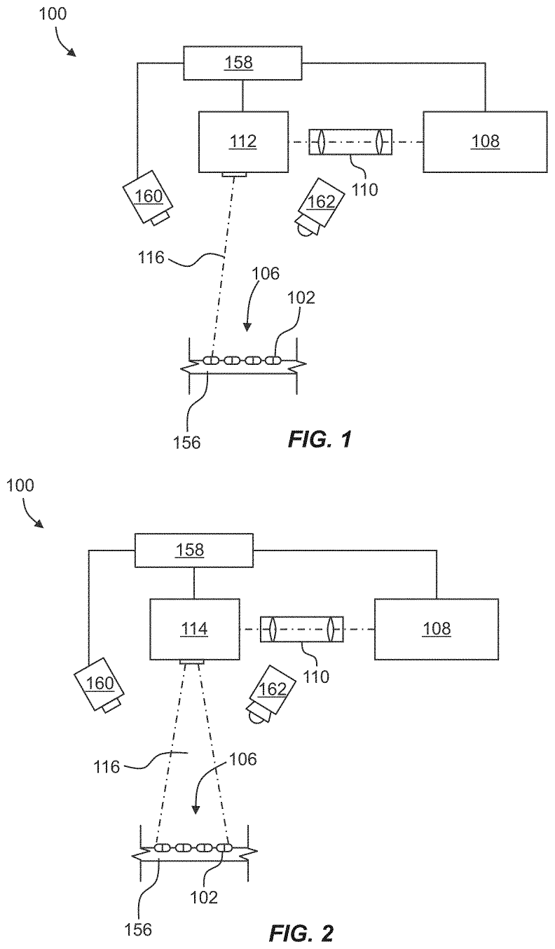

Referring to FIGS. 1 and 2, example embodiments of an ultrashort pulse laser marking apparatus for forming indelible identifiers on discreet consumable articles are illustrated generally at 100. Applicant has found that the ability to etch micro patterns on a material surface without causing thermal damage is particularly useful for pharmaceuticals (e.g., tablets and capsules), and that ultrashort pulse lasers are particularly well suited to etch precise patterns.

The apparatus 100 may comprise control electronics 158, a laser transmission element 108, a beam optics element 110, a beam controlling mechanism (such as, for example, a beam steering element 112 or beam masking element 114), and a marking zone 106. The apparatus 100 may further include an inspection camera 160 with or without a dedicated inspection light source 162. The inspection camera 160 may be configured to facilitate the verification of the application and quality of the indelible identifiers 104 disposed on the respective articles 102. The inspection camera 160 may also be configured to verify the presence, or the location and orientation, of each article 102 within the marking zone.

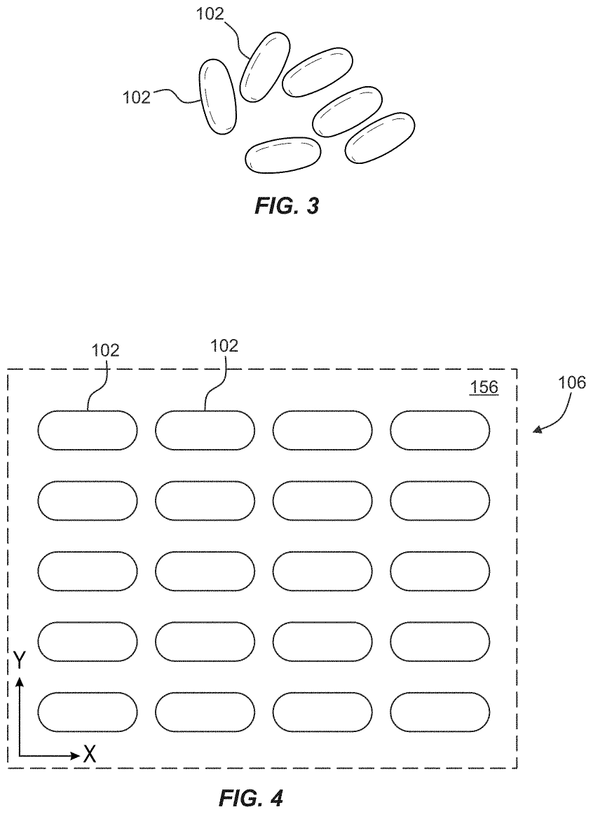

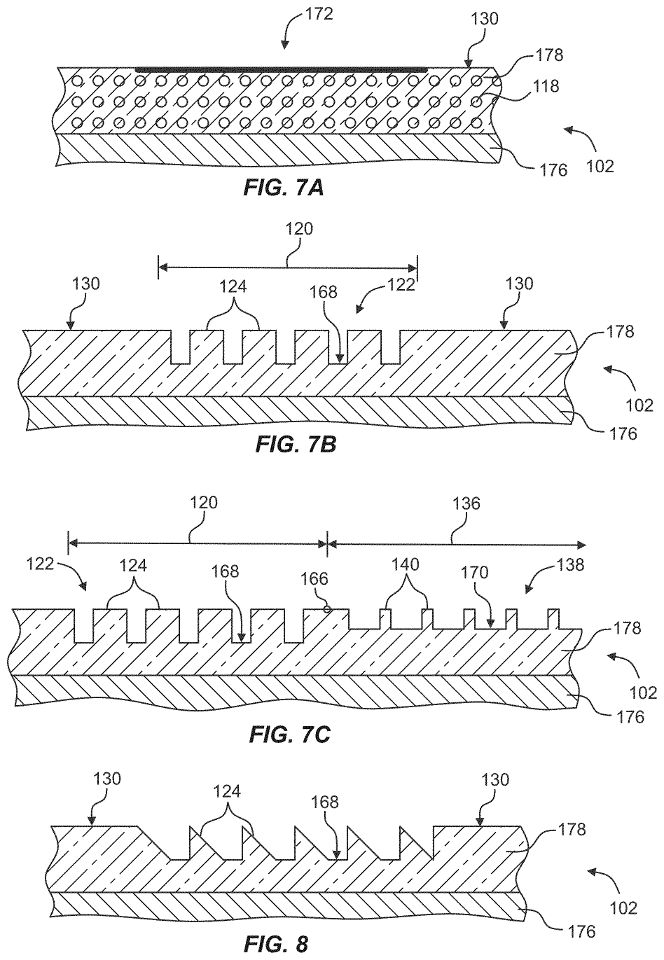

Referring to FIG. 3, a discreet consumable article 102 may be, for example, a pharmaceutical capsule or the like, such as a nutraceutical. Alternatively, the discreet consumable article 102 may be a piece of candy or other food item. Referring to FIG. 7, a consumable article 102 may have an outward layer 178 enveloping article contents 176 (for example, active medication).

The marking zone 106 may be configured to retain one or more discrete consumable articles 102. For example, referring to FIG. 3, the marking zone 106 may be defined in part by a conveyor element 156 having a plurality of pockets within which respective articles 102 are temporarily retained during the marking process. This retention may keep the respective articles in pre-determined positions and orientations within the marking zone 106 of the apparatus 100. In particular implementations of an apparatus 100, the conveyor element 156 may take the form of one or more conveyor wheels, belts or trays that may be movable continuously or intermittently through the marking zone 106.

Referring to FIGS. 1, 2 and 5, the ultrashort pulse laser transmission element 108 may be configured to transmit a beam 116 of laser energy toward the marking zone 106 to form an optically-readable indelible identifier 104 on a discrete consumable article 102. In particular implementations of the apparatus 100, the optical readability is preferably within the optical radiation portion of the electromagnetic spectrum (UV through IR). In certain implementations of the apparatus 100, the optical readability may preferably be within the human-visible portion of the electromagnetic spectrum.

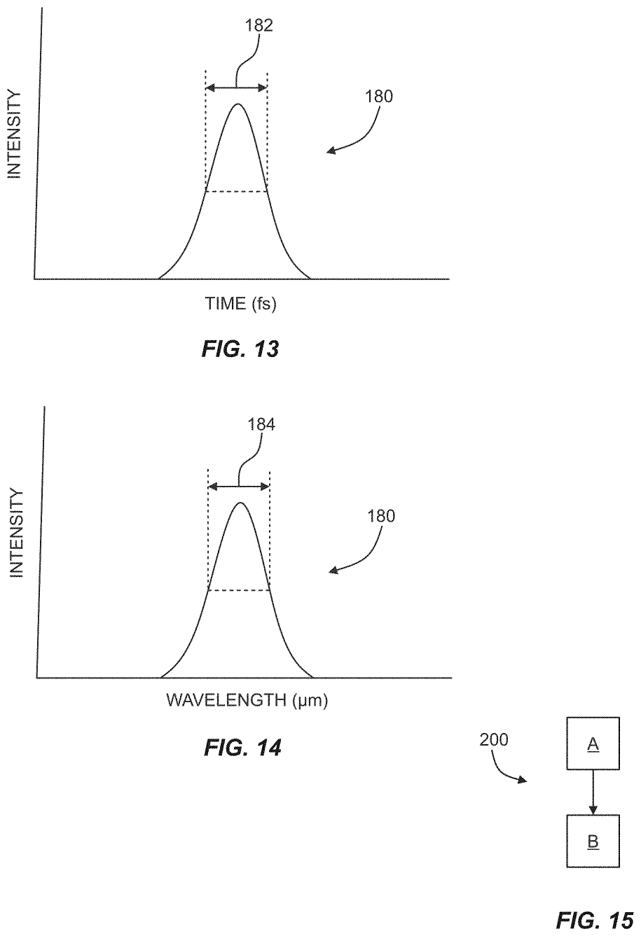

Referring to FIGS. 13 and 14, the beam 116 may preferably have a pulse duration 182 less than 10 picoseconds, and a wavelength 184 of less than 1.5 microns. In particular implementations of the apparatus 100, the pulse duration may be from 1 to 1000 femtoseconds. In certain implementations of the apparatus 100, the pulse duration 182 may be from 20 to 200 femtoseconds.

Referring to FIG. 1, the ultrashort pulse laser transmission element 108 may be coupled with a beam-steering element 112. In such implementations of the apparatus 100, the indelible identifier 104 may be generated in part by moving the beam 116 along the surface of the article 102 in, for example, X and Y directions. The beam-steering element 112 may be of a conventional type that comprises digital micro-mirrors which are movable independently of one another. Alternatively to the beam steering element 112, in certain implementations of the apparatus 100, the ultrashort pulse laser transmission element 108 may be coupled with a beam masking element 114. In such implementations, the laser beam coming from the transmission element 108 may pass through a mask which shapes the pass-through laser energy to form the entire indelible identifier on respective respective articles 102, without requiring steering of the beam 116 in the X-Y directions.

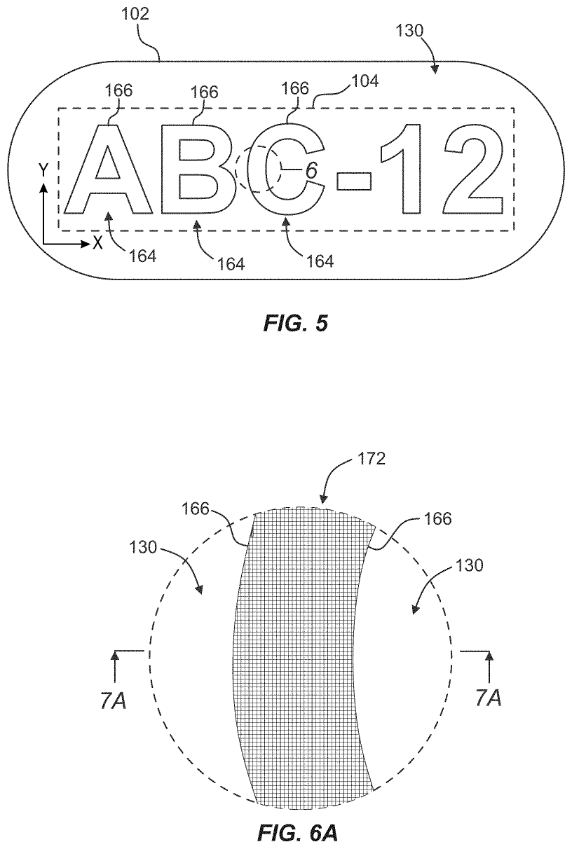

Referring to FIGS. 5, 6A and 7A, in particular implementations of the ultrashort pulse laser marking apparatus 100, the one or more consumable articles 102 may comprise a photoreactive pigment 118 (for example, in an outward layer 178) configured to undergo a color change upon exposure to the beam 116 of laser energy. In such implementations, the indelible identifier may be defined in whole or in part by the color change (for example, in the color change area 172). The photoreactive pigment 118 may comprise one or more materials selected from the group consisting of titanium dioxide, yellow iron oxide, and red iron oxide. Alternatively or in addition, the color change results from transformation of the photoreactive pigment 118 into one or more other color pigments that are approved by the United States Food and Drug Administration (FDA) for human consumption. For example, the photoreactive pigment 118 may be configured to transform into black iron oxide when exposed to the beam 116 of laser energy.

In particular implementations of the apparatus 100, the color change of the photoreactive pigment 118 may be by way of multiphoton absorption. In certain implementations of the apparatus 100, (a) the color pigment 118 may be configured to change color under UV exposure but not under IR exposure; and (b) the ultrashort pulse laser transmission element 108 may only be configured to operate in the IR range. In this regard, it is notable that UV is generally 100 nm-400 nm and IR is generally 800 nm-1500 nm. For example, TiO2 exposed to 355 nm nanosecond laser pulse changes color from white to grey or dark grey, and does not change color under exposure to 1064 nm nanosecond laser. However, the applicant finds that it will change color if an ultrashort (femtosecond) pulse of 1064 nm is used.

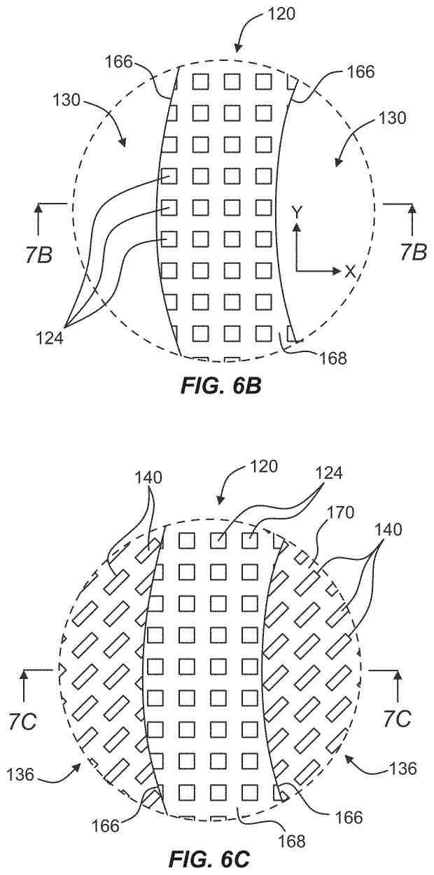

Referring to FIGS. 5 and 6B, in particular implementations of an ultrashort pulse laser marking apparatus 100, the indelible identifier 104 may be defined at least by a primary pattern field 120 having a primary array 122 of primary light interface elements 124 distributed therein. The beam 116 of laser energy may be configured to form the array of light interface elements by etching of the article 102. As a result, the apparatus 100 may preferably be configured to etch a pattern of light interface elements within one or more pattern fields on the article 102 to make the indelible identifier 104 visible responsive to directional lights.

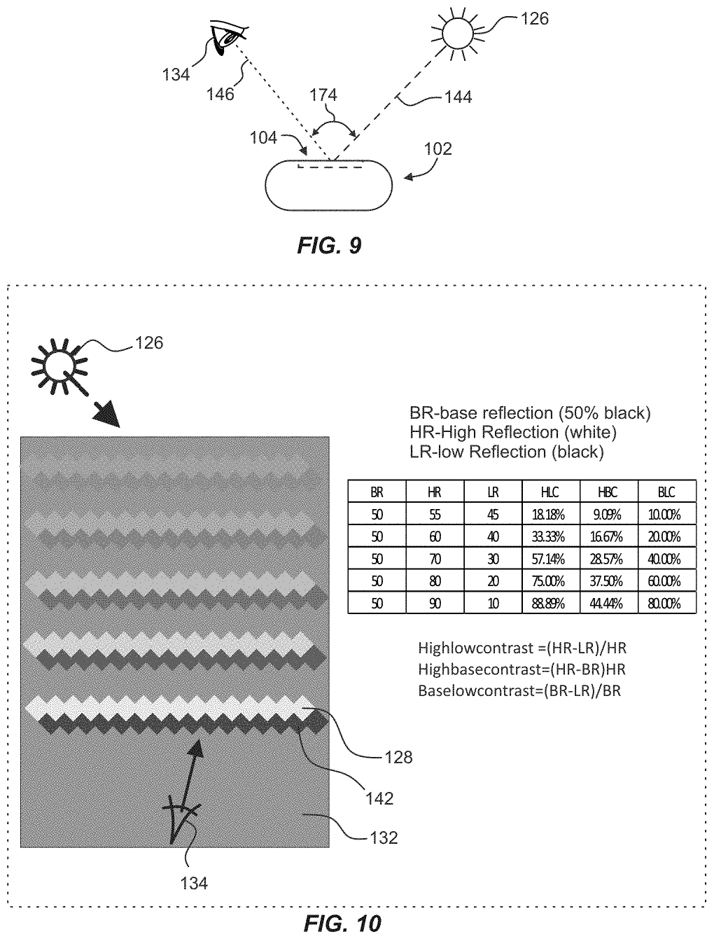

Referring to FIGS. 9, 10 and 11, the primary light interface elements 124 may be configured to interact with rays (e.g., incident rays) from a source of incoming light 126 and thereby collectively present a primary pattern reflected light intensity 128. In such implementations, the articles 102 may have a precursor surface 130 configured to interact with the source of incoming light 126 to present a baseline reflected light intensity 132. Accordingly, the optical readability may be at least in part by way of the primary pattern reflected light intensity 128 being distinguishable from the baseline reflected light intensity 132 from a viewpoint 134 outward of the article 102.

Referring to FIGS. 5 and 6C, in certain implementations of an ultrashort pulse laser marking apparatus 100, the indelible identifier 104 may be defined at least by a primary pattern field 120 having a primary array 122 of primary light interface elements 124 distributed therein. Moreover, the indelible identifier 104 may also be defined in part by a secondary pattern field 136 having a secondary array 138 of secondary light interface elements 140 distributed therein. Referring to FIGS. 9, 10 and 11, the primary light interface elements 124 may be configured to interact with rays from a source of incoming light 126 and thereby collectively present a primary pattern reflected light intensity 128. Similarly, the secondary light interface elements 140 may be configured to interact with rays from the source of incoming light 126 and thereby collectively present a secondary pattern reflected light intensity 142. In such implementations, the optical readability may be at least in part by way of the primary pattern reflected light intensity 128 being distinguishable from the secondary reflected light intensity 142 as perceived from a viewpoint 134 outward of the article 102.

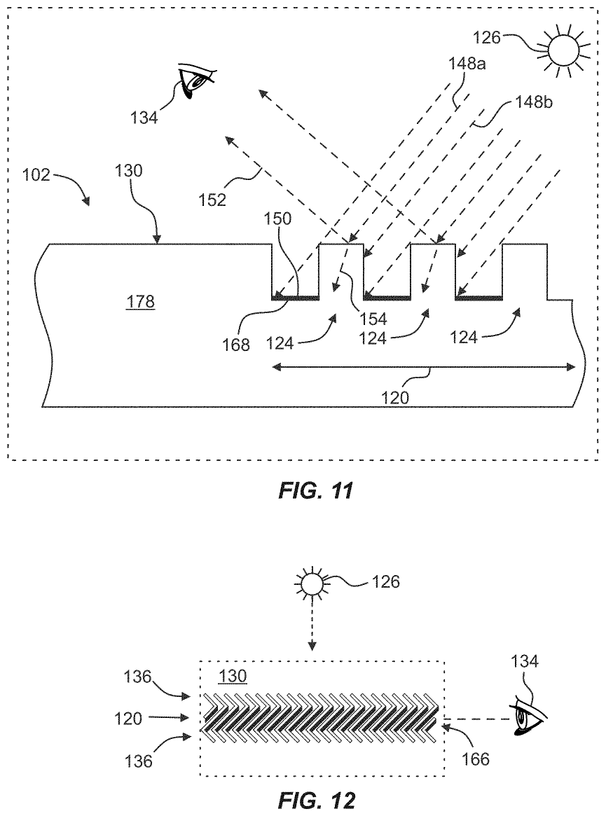

Notably, the grey-shaded image in FIG. 10 may alternatively be used to illustrate light and shadow areas produced by just the primary pattern field (e.g., arrays of raised cubes). In such case, the shadows may represent all or portions of the primary interstitial surfaces 168 where incoming light rays are at least partially blocked by the respective primary light interface elements 124 (see, for example, FIG. 11). In such implementations, the optical readability may be at least in part by way of the primary pattern reflected light intensity 128 being distinguishable from the partially-shadowed reflected light intensity of the adjacent primary interstitial surfaces 168 as perceived from a viewpoint 134 outward of the article 102.

Referring to FIGS. 9 and 11, in particular implementations of the apparatus 100 in which the indelible identifiers 104 are defined by one or more pattern fields with light interface elements, a light source axis 144 may be defined as extending between the source of incoming light 126 and the indelible identifier 104, a viewing axis 146 may be defined as extending between the indelible identifier 104 and the viewpoint 134, and the viewing axis 146 is off-angle from the light source axis 144 (for example, as defined by offset angle 174). Such an off-angle viewing may be necessary in order to increase or maximize the optical readability of certain indelible identifiers 104, depending upon the configurations of light interface elements in the respective pattern fields.

Referring to FIG. 11, the primary light interface elements 124 may be configured in part to obstruct the incoming light, reflect the incoming light, refract the incoming light, absorb the incoming light, or some combination thereof. By way of example, an incident ray 148a from a light source 126 is shown contacting a surface of a corresponding light interface element 124, where the ray is split into a refracted ray 154 and a reflected ray 152. By way of further example, an incident ray 148b shown at least partially blocked by a respective light interface element 124, thereby creating a shadow 150 on the interstitial surface 168 adjacent the opposite side of the light interface element 124.

Light interface elements may be selected to take on a variety of shapes, depending upon, for example, the desired optical properties of the indelible identifier. Moreover, referring to FIG. 7C, primary light interface elements 124 may be laterally spaced apart from one another to define primary interstitial surfaces 168 therebetween. Similarly, secondary light interface elements 140 may be laterally spaced apart from one another to define secondary interstitial surfaces 170 therebetween. In such cases, the primary interstitial surfaces 168 may be non-coplanar with the secondary interstitial surfaces 170. Light interface elements may be configured to extend from various heights with respect to their adjacent interstitial surfaces. Referring to FIGS. 6B and 6C, depending upon the particular implementation of the apparatus 100, the primary light interface elements 124 may be homogeneously shaped with respect to one another. Similarly, the secondary light interface elements 140 may be homogeneously shaped with respect to one another. Alternatively, within a particular pattern field, the light interface elements may be of various shapes, different orientations, uneven or random spacing, and the like.

In certain implementations of the apparatus 100, the light interface elements may be arranged (for example, in position or orientation) randomly throughout a majority or an entirety of the respective pattern field. In the alternative, the interface elements may be arranged in an ordered fashion throughout a majority or an entirety of the respective pattern field. For example, as illustrated in FIG. 6B, the primary light interface elements 124 may be arranged equidistantly in at least a first direction (for example, the X direction) throughout a majority or entirety of the primary pattern field 120. Also as illustrated in FIG. 6B, the primary light interface elements 124 may be arranged equidistantly in a second direction (for example, the Y direction) throughout a majority or an entirety of the primary pattern field 120. The first direction and the second direction may be perpendicular to one another. In certain implementations of the apparatus 100, the above-mentioned order may refer to long order or short order as defined along any particular direction within the X-Y plane.

Referring to FIG. 3, an indelible identifier 104 may include one or more characters 164. Each of these characters 164 may be for example, a letter, a number, a symbol, a punctuation mark, a logo, a graphic design and a machine-readable optical code some combination thereof or the like. The characters may be defined in whole or in part by respective character boundaries 166.

As illustrated in part in FIG. 12, a pattern of light interface elements may be configured to straddle the perimeter or boundary of the character, and the contrast in reflected light intensities between portions of each light interface element may define a perceived character perimeter or boundary 166. Accordingly, depending upon the implementation, the individual light interface elements do not necessarily have to be contained within the perimeter 166 of the character 164. Rather, the collection of light interface elements may cause the viewer to perceive a perimeter within an array of light interface elements.

Referring to FIG. 15, a method of using an ultrashort pulse laser marking apparatus for forming indelible identifiers on discreet consumable articles is represented generally at 200. Referring to FIGS. 1, 2 and 15, at block A of FIG. 15 the method 200 may comprise the step of providing an ultrashort pulse laser marking apparatus 100 including (a) a marking zone 106 configured to retain one or more discrete consumable articles 102; and (b) an ultrashort pulse laser transmission element 108. At block B of FIG. 15, the method 200 may further comprise transmitting, by way of the ultrashort pulse laser transmission element 108, a beam 116 of laser energy toward the marking zone 106 to form an optically-readable indelible identifier 104 on a the discrete consumable article 102. The beam 116 may have a pulse duration 182 less than 10 picosecond, and a wavelength 184 of less than 1.5 microns. The method 200 may be optionally comprise limitations that correspond to the various apparatus embodiments disclosed herein.

The following listing matches certain terminology used within this disclosure with corresponding reference numbers used in the non-limiting examples illustrated in the several figures.

100 laser marking apparatus
102 discreet consumable article
104 indelible identifier (e.g., optically-readable)
106 marking zone
108 laser transmission element
110 beam optics element
112 beam steering element
114 beam masking element
116 beam (of laser energy)
118 photoreactive pigment
120 primary pattern field
122 primary array
124 primary light interface element
126 source of incoming light
128 primary pattern reflected light intensity
130 precursor surface
132 baseline reflected light intensity
134 viewpoint
136 secondary pattern field
138 secondary array
140 secondary light interface element
142 secondary pattern reflected light intensity
144 light source axis
146 viewing axis
148a incident ray (e.g., partially reflected/refracted by light interface element)
148b incident ray (e.g., partially blocked by light interface element)
150 shadow
152 reflected ray
154 refracted ray
156 conveyor element
158 control electronics
160 inspection camera
162 inspection light source
164 character
166 character boundary
168 primary interstitial surface
170 secondary interstitial surface
172 color change area
174 offset angle
176 article contents
178 outward layer
180 laser pulse
182 pulse duration
184 pulse wavelength
200 method

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

1. An ultrashort pulse laser marking apparatus for forming indelible identifiers on discreet consumable articles, the apparatus comprising:
a marking zone configured to retain one or more discrete consumable articles; and
an ultrashort pulse laser transmission element configured to transmit a beam of laser energy toward the marking zone to form an optically-readable indelible identifier on a said discrete consumable article, the beam having a pulse duration less than 10 picosecond, and a wavelength of less than 1.5 microns.

2-7. (canceled)

8. The apparatus as defined in claim 1, wherein the one or more consumable articles comprise a photoreactive pigment configured to undergo a color change upon exposure to the beam of laser energy, and the indelible identifier is defined by the color change.

9. The apparatus as defined in claim 8, wherein the photoreactive pigment comprises one or more materials selected from the group consisting of titanium dioxide, yellow iron oxide, and red iron oxide.

10. The apparatus as defined in claim 8, wherein the color change results from transformation of the photoreactive pigment into one or more other color pigments that are approved by the United States Food and Drug Administration (FDA) for human consumption.

11. The apparatus as defined in claim 9, wherein the photoreactive pigment transforms into black iron oxide when exposed to the beam of laser energy.

12. The apparatus as defined in claim 9, wherein the color change is by way of multiphoton absorption.

13. The apparatus as defined in claim 12, wherein
(a) the color pigment is configured to change color under UV exposure but not under IR exposure; and
(b) the ultrashort pulse laser transmission element is only configured to operate in the IR range.

14. The apparatus as defined in claim 1, wherein
(a) the indelible identifier is defined at least by a primary pattern field having a primary array of primary light interface elements distributed therein, the primary light interface elements being configured to interact with rays from a source of incoming light and thereby collectively present a primary pattern reflected light intensity,
(b) the article has a precursor surface configured to interact with the source of incoming light to present a baseline reflected light intensity, and
(c) the optical readability is at least in part by way of the primary pattern reflected light intensity being distinguishable from the baseline reflected light intensity from a viewpoint outward of the article.

15. The apparatus as defined in claim 1, wherein
(a) the indelible identifier is defined in part by a primary pattern field having a primary array of primary light interface elements distributed therein, the primary light interface elements being configured to interact with rays from a source of incoming light and thereby collectively present a primary pattern reflected light intensity,
(b) the indelible identifier is defined in part by a secondary pattern field having a secondary array of secondary light interface elements distributed therein, the secondary light interface elements being configured to interact with rays from the source of incoming light and thereby collectively present a secondary pattern reflected light intensity, and
(c) the optical readability is at least in part by way of the primary pattern reflected light intensity being distinguishable from the secondary reflected light intensity from a viewpoint outward of the article.

16. The apparatus as defined in claim 14, wherein
(a) a light source axis is defined as extending between the source and the indelible identifier,
(b) a viewing axis is defined as extending between the indelible identifier and the viewpoint, and
(c) the viewing axis is off-angle from the light source axis.

17. The apparatus as defined in claim 14, wherein the primary light interface elements are configured in part to obstruct the incoming light.

18. The apparatus as defined in claim 14, wherein the primary light interface elements are configured in part to reflect the incoming light.

19. The apparatus as defined in claim 14, wherein the primary light interface elements are configured in part to refract the incoming light.

20. The apparatus as defined in claim 14, wherein the primary light interface elements are configured in part to absorb the incoming light.

21. The apparatus as defined in claim 14, wherein the indelible identifier includes one or more characters selected from the group consisting of a letter, a number, a symbol, a punctuation mark, a logo, a graphic design and a machine-readable optical code.

22. The apparatus as defined in claim 14, wherein the primary light interface elements are homogeneously shaped with respect to one another.

23. The apparatus as defined in claim 14, wherein the primary light interface elements are arranged randomly throughout a majority or an entirety of the primary pattern field.

24. The apparatus as defined in claim 14, wherein the primary light interface elements are arranged equidistantly in a first direction throughout a majority or entirety of the primary pattern field.

25. The apparatus as defined in claim 24, wherein the primary light interface elements are arranged equidistantly in a second direction throughout a majority or an entirety of the primary pattern field.

26. The apparatus as defined in claim 25, wherein the first direction and the second direction are perpendicular to one another.

27. The apparatus as defined in claim 15, wherein
(a) the primary light interface elements are spaced apart from one another to define primary interstitial surfaces therebetween;
(b) the secondary light interface elements are spaced apart from one another to define secondary interstitial surfaces therebetween; and
(c) the primary interstitial surfaces are non-coplanar with the secondary interstitial surfaces.

28. The apparatus as defined in claim 1, wherein the optical readability is within the optical radiation portion of the electromagnetic spectrum.

29. The apparatus as defined in claim 28, wherein the optical readability is within the human-visible portion of the electromagnetic spectrum.

30. The apparatus as defined in claim 14, wherein the beam of laser energy is configured to form the array of light interface elements by etching of the article.

31-60. (canceled)

61. The apparatus as defined in claim 15, wherein
(a) a light source axis is defined as extending between the source and the indelible identifier,
(b) a viewing axis is defined as extending between the indelible identifier and the viewpoint, and
(c) the viewing axis is off-angle from the light source axis.

62. The apparatus as defined in claim 15, wherein the primary light interface elements are configured in part to obstruct the incoming light.

63. The apparatus as defined in claim 15, wherein the primary light interface elements are configured in part to reflect the incoming light.

64. The apparatus as defined in claim 15, wherein the primary light interface elements are configured in part to refract the incoming light.

65. The apparatus as defined in claim 15, wherein the primary light interface elements are configured in part to absorb the incoming light.

66. The apparatus as defined in claim 15, wherein the indelible identifier includes one or more characters selected from the group consisting of a letter, a number, a symbol, a punctuation mark, a logo, a graphic design and a machine-readable optical code.

67. The apparatus as defined in claim 15, wherein the primary light interface elements are homogeneously shaped with respect to one another.

68. The apparatus as defined in claim 15, wherein the primary light interface elements are arranged randomly throughout a majority or an entirety of the primary pattern field.

69. The apparatus as defined in claim 15, wherein the primary light interface elements are arranged equidistantly in a first direction throughout a majority or entirety of the primary pattern field.

70. The apparatus as defined in claim 69, wherein the primary light interface elements are arranged equidistantly in a second direction throughout a majority or an entirety of the primary pattern field.

71. The apparatus as defined in claim 70, wherein the first direction and the second direction are perpendicular to one another.

72. The apparatus as defined in claim 15, wherein the beam of laser energy is configured to form the array of light interface elements by etching of the article.
